(19) United States
(12) Patent Application Publication
(10) Pub. No.: US 2022/0089629 A1
(43) Pub. Date: Mar. 24, 2022

(54) AMORPHOUS SIALYLATED OLIGOSACCHARIDES

(71) Applicant: GLYCOM A/S, Hørsholm (DK)

(72) Inventors: Benjámin PODÁNYI, Dunakeszi (HU); Martin MATWIEJUK, Hamburg (DE); Dóra MOLNÁR-GÁBOR, Budapest (HU)

(21) Appl. No.: 17/310,039

(22) PCT Filed: Jan. 16, 2020
(86) PCT No.: PCT/IB2020/050332

§ 371 Date: Jul. 13, 2021

(30) Foreign Application Priority Data
Jan. 16, 2019 (DK) PA 2019 00063

(51) Int. Cl. C07H 3/06; C07H 1/00

(52) U.S. Cl. CPC C07H 3/06; C07H 1/00

(57) ABSTRACT

It is provided i) an amorphous carbohydrate with improved chemical stability and/or physical features, ii) a method for producing an amorphous carbohydrate with improved chemical stability and/or physical features, and iii) a method for improving the chemical stability and/or the physical features of an amorphous carbohydrate.

### FIELD OF THE INVENTION

The present invention relates to amorphous, preferably spray-dried or freeze-dried sialylated oligosaccharides, advantageously human milk oligosaccharides, and a method for increasing the stability of amorphous sialylated oligosaccharides.

### BACKGROUND OF THE INVENTION

In recent years, the manufacture and commercialization of complex carbohydrates including naturally secreted oligosaccharides have increased significantly due to their roles in numerous biological processes occurring in living organisms. Secreted oligosaccharides such as human milk oligosaccharides (HMOs) are carbohydrates which have gained much interest in recent years and are becoming important commercial targets for nutrition and therapeutic industries. In particular, the synthesis of these HMOs has increased significantly due to the role of HMOs in numerous biological processes occurring in humans. The great importance of HMOs is directly linked to their unique biological activities such as antibacterial, antiviral, immune system and cognitive development enhancing activities. Human milk oligosaccharides are found to act as prebiotics in the human intestinal system helping to develop and maintain the intestinal flora. Furthermore, they have also proved to be anti-inflammatory, and therefore these compounds are attractive components in the nutritional industry for the production of, for example, infant formulas, infant cereals, clinical infant nutritional products, toddler formulas, or as dietary supplements or health functional food for children, adults, elderly or lactating women, both as synthetically composed and naturally occurring compounds and salts thereof. Likewise, the compounds are also of interest in the medicinal industry for the production of therapeutics due to their prognostic use as immunomodulators. To date, the structures of more than 140 HMOs have been determined (see Urashima et al.: Milk Oligosaccharides, Nova Biomedical Books, New York, 2011; Chen Adv. Carbohydr. Chem. Biochem. 72, 113 (2015)), and considerably more are probably present in human milk. Sialylated HMOs are thought to have significant health benefits for the neonate, because of their roles in supporting resistance to pathogens, gut maturation, immune function and cognitive development (ten Bruggencate et al. Nutr. Rev. 72, 377 (2014)).

Sialylated human milk oligosaccharides mainly occur as amorphous solids in isolated form, see e.g. Fierfort et al. J. Biotechnol. 134, 261 (2008), Drouillard et al. Carbohydr. Res. 345, 1394 (2010), WO 2017/152918 or US 2018/0002363. Only some salts of 6′-sialyllactose (6′-SL) and a sodium salt of 3′-sialyllactose (3′-SL) have been described as crystalline material (see WO 2010/116317, EP-A-3378868, WO 2017/195743).

The properties of a crystalline and an amorphous substance are different. While the amorphous form of a substance is, in general, expected to show better solubility and bioavailability, its crystalline counterpart is chemically and physically more stable and less hygroscopic (Bauer J. Valid. Technol. 63 (Summer 2009)).

WO 2013/185780 discloses a method for enhancing the stability of 6′-SL so that the aqueous solution of 6′-SL is spray-dried to provide the 6′-SL with a glass transition temperature (T_{g}) of 84° C.

Nevertheless, there is a need to improve the chemical stability and/or the physical features of an amorphous sialylated oligosaccharide, preferably sialylated HMO, while its beneficial properties are maintained.

### SUMMARY OF THE INVENTION

It is the object of the present invention to provide i) an amorphous sialylated oligosaccharide with improved chemical stability and/or physical features, ii) a method for producing a sialylated oligosaccharide with improved chemical stability and/or physical features, and iii) a method for improving the chemical stability and/or the physical features of a sialylated oligosaccharide.

Accordingly, one aspect of the invention relates to an amorphous sialylated oligosaccharide or its salt, wherein the pH of said sialylated oligosaccharide, when measured in its 5 w/w % aqueous solution, is around 3.9-6.0, preferably 3.9-5.3. Also preferably, the amorphous sialylated oligosaccharide is a spray-dried or a freeze-dried substance.

Another aspect of the invention relates to a method for making an amorphous sialylated oligosaccharide or its salt, comprising the steps of
a) preparing an aqueous solution of said sialylated oligosaccharide,
b) if necessary, adding an acidic or a basic non-carbohydrate component to the aqueous solution obtained in step a) so that the pH is not higher than 5.6, preferably lower than 5.0, then
c) solidifying, preferably spray-drying or freeze-drying, the aqueous solution obtained in step a) or the pH adjusted solution obtained in step b), thereby obtaining an amorphous sialylated oligosaccharide, wherein the pH of said sialylated oligosaccharide, when measured in its 5 w/w % aqueous solution, is not more than around 6.0, preferably not more than around 5.3.

Another aspect of the invention relates to a method for improving the chemical stability and/or the physical properties of an amorphous sialylated oligosaccharide, the method comprising solidifying, preferably spray-drying or freeze-drying, an aqueous solution of the sialylated oligosaccharide or its salt, the pH of the aqueous solution is not lower than 3.6 and not higher than 5.6, preferably lower than 5.0. The above method provides an amorphous sialylated oligosaccharide or its salt, the pH of which, when measured in its 5 w/w % aqueous solution, is around 3.9-6.0, preferably around 3.9-5.3, and showing improved chemical stability and/or the physical properties.

Another aspect of the invention is a method for preventing or reducing the isomerization in an amorphous sialylated oligosaccharide or its salt from aldose to ketose, the method comprising spray-drying or freeze-drying an aqueous solution of the sialylated oligosaccharide, the pH of the aqueous solution is not higher than 5.6, preferably lower than 5.0. The above method provides an amorphous sialylated oligosaccharide or its salt, the pH of which, when measured in its 5 w/w % aqueous solution, is not higher than around 6.0, preferably not higher than around 5.3, that show s a reduced aldose-ketose isomerization.

Another aspect of the invention is an amorphous sialylated oligosaccharide or its salt obtained or obtainable by a method comprising the steps:
a) preparing an aqueous solution of said sialylated oligosaccharide,
b) if necessary, adding an acidic or a basic non-carbohydrate component to the aqueous solution obtained in step a) so that the pH is not higher than 5.6, preferably lower than 5.0, then
c) solidifying, preferably spray-drying or freeze-drying, the aqueous solution obtained in step a) or the pH adjusted solution obtained in step b), thereby obtaining an amorphous sialylated oligosaccharide, wherein the pH of said sialylated oligosaccharide, when measured in its 5 w/w % aqueous solution, is not more than around 6.0, preferably not more than around 5.3.

Preferably, the pH is adjusted in step b) so that is not lower than 3.6 and not higher than 5.6, preferably not lower than 3.6 and lower than 5.0.

### BRIEF DESCRIPTION OF THE FIGURE

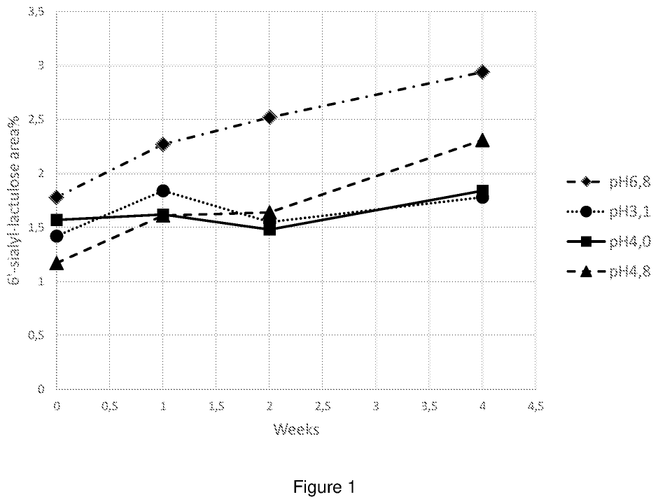

The invention will be described in further detail hereinafter with reference to the accompanying FIG. 1, which shows the amount of 6′-sialyllactulose formed in freeze-dried 6′-SL samples having various pH during accelerated heat stability test.

### DETAILED DESCRIPTION OF THE INVENTION

### Terms and Definitions

The term “sialylated oligosaccharide” means a sugar polymer consisting of at least two, preferably from three to eight, more preferably from three to six, monosaccharide units, at least one of which is sialic acid (N-acetyl neuraminic acid, Neu5Ac). The monosaccharide unit(s) other than sialic acid in a sialylated is a sugar of 5-9 carbon atoms that is an aldose (e.g. D-glucose, D-galactose, D-mannose, D-ribose, D-arabinose, L-arabinose, D-xylose, etc.), a ketose (e.g. D-fructose, D-sorbose, D-tagatose, etc.), a deoxysugar (e.g. L-rhamnose, L-fucose, etc.), a deoxy-aminosugar (e.g. N-acetylglucosamine, N-acetylmannosamine, N-acetylgalactosamine, etc.), an uronic acid, a ketoaldonic acid (e.g. sialic acid) or a deoxyamino sugar with free amino group (e.g. glucosamine), provided that the monosaccharide unit at the reducing end of the sialylated oligosaccharide is an aldose. The tri- or higher oligosaccharide can have a linear or branched structure containing monosaccharide units that are linked to each other by interglycosidic linkages.

The term “sialylated human milk oligosaccharide”, “sialylated HMO” or “acidic HMO” means a complex carbohydrate found in human breast milk (Urashima et al.: Milk Oligosaccharides, Nova Medical Books, N Y, 2011; Chen Adv. Carbohydr. Chem. Biochem. 72, 113 (2015)) that comprises a sialic acid. The HMOs have a core structure being a lactose unit at the reducing end that is elongated by one or more β-N-acetyl-lactosaminyl and/or one or more β-lacto-N-biosyl units, and which core structures is substituted by an α-sialyl moiety and optionally by an α-L-fucopyranosyl. In a sialylated HMO, the sialyl group is linked to a terminal galactose with an α2,3- or an α2,6-linkage, or to an N-acetylglucosamine with an α2,6-linkage:

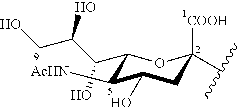

Examples of acidic HMOs include 3′-sialyllactose (3′-SL), 6′-sialyllactose (6′-SL), 3-fucosyl-3′-sialyllactose (FSL), LST a (NeuAca2-3Galβ1-3GlcNAcβ1-3Galβ1-4Glc), fucosyl-LST a (FLST a, NeuAcα2-3Galβ1-3[Fucα1-4]GlcNAcβ1-3Galaβ1-4Glc), LST b (Galβ1-3[NeuAca2-6]GlcNAcβ1-3Galβ1-4Glc), fucosyl-LST b (FLST b, Fucα1-2Galβ1-3[NeuAca2-6]GlcNAcβ1-3Galβ1-4Glc), LST c (NeuAca2-6Galβ1-4GlcNAcβ1-3Galβ1-4Glc), fucosyl-LST c (FLST c, NeuAca2-6Galβ1-4GlcNAcβ1-3Galβ1-4[Fucα1-3]Glc), sialyl-lacto-N-hexaose (SLNH, Galβ1-3GlcNAcβ1-3[NeuAca2-6Galβ1-4GlcNAcβ1-6]Galβ1-4Glc), sialyl-lacto-N-neohexaose I (SLNnH-I, Galβ1-4GlcNAcβ1-3[NeuAca2-6Galβ1-4GlcNAcβ1-6]Galβ1-4Glc), sialyl-lacto-N-neohexaose II (SLNnH-II, NeuAcα2-6Galβ1-4GlcNAcβ1-3[Galβ1-4GlcNAcβ1-6]Galβ1-4Glc) and disialyl-lacto-N-tetraose (DS-LNT, NeuAca2-3Galβ1-3[NeuAca2-6]GlcNAcβ1-3Galβ1-4Glc).

The term “acid addition salt of a sialylated oligosaccharide”, “acid addition salt of a sialylated HMO”, “salt of a sialylated oligosaccharide” or “salt of a sialylated HMO” means an associated ion pair consisting of the negatively charged acid residue of the sialylated oligosaccharide or HMO and a cation in any stoichiometric proportion. The cation is typically an inorganic (metal) cation. In the acid addition salt of the sialylated oligosaccharide, preferably sialylated HMO, more preferably 3′-SL or 6′-SL, the salt forming cation is, in one embodiment, an alkali metal ion (M⁺), an alkali earth metal ion (M²⁺) or a mixture thereof, such as Na⁺, K⁺ or Ca²⁺, preferably Na⁺.

The term “around” means, in one embodiment, ±10% deviation from the value indicated, or in another embodiment, ±5% deviation.

When a pH value is referred to, it is measured by a pH-meter conventionally.

Amorphous Sialylated Oligosaccharides with Improved Features

Although an amorphous compound, especially a hydrophilic compound, generally shows better water solubility, higher dissolution rate and better bioavailability, which are key features in product development in the pharma and food industry, its shelf-life is expected to be shorter than that of the corresponding crystalline form. The amorphous compounds tend to undergo chemical degradation/transformation in higher degree than their crystalline form, and their physical appearance may change disadvantageously, e.g. due to agglutination. The present inventors noticed that in certain samples of amorphous (spray-dried or freeze-dried) sialylated oligosaccharides with aldose reducing terminal, upon prolonged storage, a new carbohydrate type by-product appeared, the amount of which in the sample increased as a function of time. As a result of careful analysis, this contamination proved to be a rearranged ketose derivative of the reducing aldose oligosaccharide, which rearrangement can be illustrated on an aldohexose as follows:

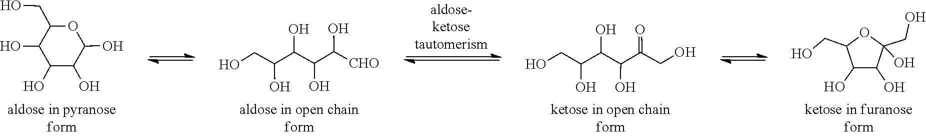 [SMILES: OCC1OC(O)C(O)C(O)C1O ; O=CC(O)C(O)C(O)C(O)CO ; O=C(CO)C(O)C(O)C(O)CO ; OCC1OC(O)(CO)C(O)C1O]

The present inventors surprisingly found that the above rearrangement does not occur, or at least occurs in a significantly lower or reduced degree, in amorphous state of the sialylated oligosaccharide, if the amorphous, e.g. spray-dried or freeze-dried, sialylated oligosaccharide is prepared from its aqueous solution in which the pH is carefully adjusted. In this regard the chemical stability of the spray-dried or freeze-dried material can be substantially improved.

Accordingly, the first aspect of the invention is to provide an amorphous sialylated oligosaccharide, wherein the pH of said amorphous sialylated oligosaccharide, when measured in its 5 w/w % aqueous solution, is not more than 6, preferably around 3.9-6.0, more preferably not more than 5.3, even more preferably 3.9-5.3.

The amorphous sialylated oligosaccharide, in one embodiment, is a spray-dried material, in other embodiment, is a freeze-dried substance.

Preferably, in one embodiment, the monosaccharide unit at the reducing end of the sialylated oligosaccharide is glucose, more preferably a galactose unit is attached to said glucose with a β1-4 interglycosidic linkage to form a lactose moiety.

In a preferred embodiment, the sialylated oligosaccharide is a tri- or higher oligosaccharide, advantageously having a lactose moiety at the reducing end.

Particularly, the tri- or higher sialylated oligosaccharide comprising a lactose moiety at the reducing end is a sialylated human milk oligosaccharide (HMO). The sialylated HMO is preferably a tri- to octasaccharide HMO, more preferably a tri-, tetra-, penta- or hexasaccharide HMO. More preferably, a trisaccharide sialylated HMO can be selected from the group consisting of 3′-sialyllactose (3′-SL) and 6′-sialyllactose (6′-SL); a tetrasaccharide sialylated HMO is 3-fucosyl-3′-sialyllactose (FSL); a pentasaccharide sialylated HMO can be selected from the group consisting of LST a (NeuAca2-3Galβ1-3GlcNAcβ1-3Galβ1-4Glc), LST b (Galβ1-3[NeuAca2-6]GlcNAcβ1-3Galβ1-4Glc) and LST c (NeuAca2-6Galβ1-4GlcNAcβ1-3Galβ1-4Glc); a hexasaccharide sialylated HMO can be selected from the group consisting of fucosyl-LST a (FLST a, NeuAca2-3Galβ1-3[Fucα1-4]GlcNAcβ1-3Galβ1-4Glc), fucosyl-LST b (FLST b, Fucα1-2Galβ1-3[NeuAca2-6]GlcNAcβ1-3Galβ1-4Glc), fucosyl-LST c (FLST c, NeuAca2-6Galβ1-4GlcNAcβ1-3Galβ1-4[Fucα1-3]Glc) and disialyl-lacto-N-tetraose (DS-LNT, NeuAca2-3Galβ1-3[NeuAca2-6]GlcNAcβ1-3Galβ1-4Glc).

In one embodiment, the amorphous, preferably the spray-dried or freeze-dried, sialylated oligosaccharide according to the present invention may comprise more than one sialylated oligosaccharide or HMO, such as two, three, four or five sialylated oligosaccharides or HMOs.

In other embodiment, the amorphous, preferably the spray-dried or freeze-dried, sialylated oligosaccharide according to the present invention may comprise one or more non-sialylated neutral oligosaccharide; preferably, the amorphous, preferably the spray-dried or freeze-dried, sialylated HMO according to the present invention may comprise one or more non-sialylated neutral HMO, such as one or more of 2′-FL, 3-FL, DFL, LNT and LNnT. In a preferred embodiment, the amorphous, preferably the spray-dried or freeze-dried, sialylated oligosaccharide according to the present invention consists or consists essentially of one or two sialylated oligosaccharides; more preferably, the spray-dried or freeze-dried, sialylated HMO according to the present invention consists or consists essentially of one or two sialylated HMO.

Forced stability comparison tests at high temperatures showed that the careful adjustment of the pH of the aqueous solution of the sialylated oligosaccharide, preferably sialylated HMO, before solidification into an amorphous material, e.g. by spray-drying or freeze-drying, significantly reduced the formation of the rearranged ulose in solid (amorphous) state. The pH of the claimed amorphous, for example spray-dried or freeze-dried, sialylated oligosaccharide or HMO in its 5 w/w % aqueous solution should be not higher than around 6.0. A pH higher than this is very close to the neutral region, at which, according to the comparison tests, the aldose-ketose rearrangement can more easily occur. Therefore, the aldose-ketose isomerization in an amorphous sialylated oligosaccharide can be reduced, minimalized or even prevented, when said sialylated oligosaccharide is produced so that the solidified, e.g. by spray-drying or freeze-drying, sialylated oligosaccharide has a pH of not higher than 6.0 when it is measured in its 5 w/w % aqueous solution, such as not more than around 4.5, 5.1 or 5.6, for example between around 3.5-6, 3.5-5.6, 3.5-5.1, 3.5-4.5, 3.9-6, 3.9-5.6, 3.9-5.1, 3.9-4.5, 4.2-5.6, 4.5-5.6, 4.5-6.0, 4.2-5.1, 4.5-5.1, 5.1-6.0, 5.1-5.6 or 5.6-6.0. Preferably, the pH of the claimed amorphous, for example spray-dried or freeze-dried, sialylated oligosaccharide or HMO in its 5 w/w % aqueous solution should be not higher than around 5.3, such as not more than around 4.5 or 5.1, for example between around 3.5-5.1, 3.5-4.8, 3.5-4.5, 3.5-4.2, 3.5-3.9, 3.9-5.1, 3.9-4.8, 3.9-4.5, 3.9-4.2, 4.2-5.1, 4.2-4.8, 4.2-4.5, 4.5-5.1 or 4.5-4.8. More preferably, the pH of the claimed amorphous, for example spray-dried or freeze-dried, sialylated oligosaccharide or HMO in its 5 w/w % aqueous solution should be not higher than around 5.1, such as not more than around 4.5 or 4.8, for example between around 3.5-5.1, 3.5-4.8, 3.5-4.5, 3.5-4.2, 3.5-3.9, 3.9-5.1, 3.9-4.8, 3.9-4.5, 3.9-4.2, 4.2-5.1, 4.2-4.8, 4.2-4.5, 4.5-5.1 or 4.5-4.8. Even more preferably, the pH of the claimed amorphous, for example spray-dried or freeze-dried, sialylated oligosaccharide or HMO in its 5 w/w % aqueous solution should be not higher than around 4.8, such as not more than around 4.5, for example between around 3.5-4.8, 3.5-4.5, 3.5-4.2, 3.5-3.9, 3.9-4.8, 3.9-4.5, 3.9-4.2, 4.2-4.8, 4.2-4.5 or 4.5-4.8.

In addition, forced stability comparison tests at high temperatures showed that the lower the pH of the aqueous solution of the amorphous sialylated oligosaccharide or HMO, the higher the possibility of leaving the sialyl residue from its structure in solid (amorphous) state. In this regard, the careful adjustment of the pH of the aqueous solution of the sialylated oligosaccharide before solidification into an amorphous material, e.g. by spray-drying or freeze-drying, minimizes the overall by-product formation (that is the rearranged ulose and the hydrolysed product) in the solid (amorphous, e.g. spray-dried or freeze-dried) state of said sialylated oligosaccharide or HMO. Accordingly, it is advisable that the pH of the amorphous, for example spray-dried or freeze-dried, sialylated oligosaccharide or HMO, when it is measured in its 5 w/w % aqueous solution, is not lower than around 3.9. A pH lower than around 3.9, even in solid state, seems to initiate the an acid catalysed degradation of the oligosaccharide by hydrolysing at least one of the interglycosidic linkages, preferably the sialoside linkage. Therefore, it is preferred when the pH of the 5 w/w % solution of the sialylated oligosaccharide after solidification to an amorphous material is between around 3.9-6.0, such as 3.9-5.6, 3.9-5.1, 4.2-5.6, 4.5-5.6, 4.5-6.0, 4.2-5.1, 4.5-5.1, 5.1-6.0, 5.1-5.6 or 5.6-6.0, more preferably between around 3.9-5.3, such as 3.9-5.1, 4.2-5.1, 4.5-5.1, 4.8-5.1, 4.2-5.3, 4.5-5.3, 4.8-5.3, 3.9-4.8, 4.2-4.8, 4.5-4.8, 3.9-4.5, 4.2-4.5 or 3.9-4.2, even more preferably between around 3.9-5.1, such as 4.2-5.1, 4.5-5.1, 4.8-5.1, 3.9-4.8, 4.2-4.8, 4.5-4.8, 3.9-4.5, 4.2-4.5 or 3.9-4.2, particularly between around 3.9-4.8, such as 4.2-4.8, 4.5-4.8, 3.9-4.5, 4.2-4.5 or 3.9-4.2, to improve the chemical stability and/or the physical properties of said amorphous sialylated oligosaccharide, for example by reducing or minimizing the content of degraded (e.g. hydrolysed) and/or transformed (e.g. rearranged, such as ulose rearranged) by-products in the amorphous sialylated oligosaccharide under prolonged storage.

A second aspect of the invention relates to a method for making an amorphous sialylated oligosaccharide or HMO, preferably that according to the first aspect, comprising the steps of
a) preparing an aqueous solution of said sialylated oligosaccharide or HMO,
b) if necessary, adding an acidic or a basic non-carbohydrate component to the aqueous solution obtained in step a) so that the pH is not higher than 5.6, preferably lower than 5.0, then
c) solidifying, preferably spray-drying or freeze-drying, the aqueous solution obtained in step a) or the pH adjusted solution obtained in step b),

thereby obtaining an amorphous sialylated oligosaccharide or HMO, wherein the pH of said sialylated oligosaccharide or HMO, when measured in its 5 w/w % aqueous solution, is not more than around 6.0, preferably not more than around 5.3.

In step b), the phrase “if necessary” refers to the scenario when the pH of the solution obtained in step a) is higher than 5.6 and the pH must be lowered to 5.6 or below by adding an acidic component; preferably, if the pH is not lower than 5.0, pH must be lowered below 5.0. Furthermore, the addition of a base may be necessary if the solution obtained in step a) is too acidic. It follows that if the pH of the solution obtained in step a) is higher than 5.6, step b) is compulsory and step c) is solidifying the so-obtained solution after step b), otherwise step b) is optional; preferably, if the pH of the solution obtained in step a) is not lower than 5.0, step b) is compulsory and step c) is solidifying the so-obtained solution after step b), otherwise step b) is optional. If the pH of the solution obtained in step a) is not higher than 5.6, preferably lower than 5.0, then step b) can be skipped and step c) is solidifying the solution obtained in step a).

In this regard, the method according to the second aspect comprises the steps of:
i) preparing an aqueous solution of a sialylated oligosaccharide or HMO,
ii) checking the pH of the solution obtained in step i),
iiia) if said pH is higher than 5.6, an acidic non-carbohydrate component is added to lower the pH to 5.6 or below, preferably if said pH is not lower than 5.0, an acidic non-carbohydrate component is added to lower the pH below 5.0,
iiib) if said pH is not higher than 5.6, an acidic or a basic non-carbohydrate component may optionally be added so that the pH remains not higher than 5.6; preferably, if said pH is lower than 5.0, an acidic or a basic non-carbohydrate component may optionally be added so that the pH remains lower than 5.0, and
iv) solidifying, preferably spray-drying or freeze-drying, the aqueous solution obtained in step iiia) or step iiib).

In one embodiment, an amorphous sialylated oligosaccharide according to the first aspect can be made, wherein
if necessary in step b), an acidic or a basic non-carbohydrate component is added to the aqueous solution obtained in step a) so that the pH is not lower than 3.6 and not higher than 5.6, preferably lower than 5.0, or
in step iiia), an acidic non-carbohydrate component is added to set the pH between 3.6 and 5.6, preferably between 3.6 and lower than 5.0, or
in step iiib), an acidic or a basic non-carbohydrate component may optionally be added so that the pH remains or is set between 3.6 and 5.6, preferably between 3.6 and lower than 5.0.

In step a) or step i) of the method, an aqueous solution of the sialylated oligosaccharides is made in a conventional manner. It is preferred when the solution is clear and sialylated oligosaccharide is completely dissolved. The sialylated oligosaccharides, before addition of water to it/or adding it to water, may be in any solid form (for example, crystalline, amorphous [precipitated, freeze-dried, spray-dried] or mixture thereof), in syrupy form or even in an aqueous solution. The sialylated oligosaccharide, before conducting step a) or step i), may contain some amounts or a residual amount of (volatile) organic solvent(s), because those solvents are or can be substantially removed in step c) or step iv) of the method and will not be comprised in the amorphous final substance obtainable. The concentration of the sialylated oligosaccharide aqueous solution made in step a) or step i) is not critical and can range a broad concentration interval when the pH is acidic. The concentration of the sialylated oligosaccharide or HMO in its aqueous solution does not really affect the measured pH, the concentration dependence of the pH it is within approximately a ±0.3 pH unit range, the closer to the pKₐ value of the sialylated oligosaccharide the smaller the expected change. A convenient concentration range to work with is that being not too diluted and not too concentrated, for example between 1-60 w/w %.

In step b) or step iiib) of the method, the pH adjustment of the solution obtained in step a) or step i), respectively, may be necessary. The sialylated oligosaccharide itself is an acidic compound, therefore its aqueous solution practically has a pH of less than 7, more precisely it depends on the ratio of its acidic and the complementary basic (salt) form in the solution. Thus, either an acidic substance, preferably acidic non-carbohydrate component, or a basic substance, preferably a basic non-carbohydrate component, may be added to set up the required pH if one wishes to deviate from the compound's original pH.

In any of the cases in step b), iiia) or iiib), the acidic or the basic substance is added slowly, preferably under stirring, to the aqueous solution of the sialylated oligosaccharide, and the pH is continuously checked e.g. by a pH-meter. If the required pH is achieved, the addition of acid or base is terminated. The concentration of the sialylated oligosaccharide in its aqueous solution when the pH adjustment is done preferably between 1-60 w/w %, for practical reason.

The acidic non-carbohydrate component, added in step iiia) or optionally added in step b) or iiib), is typically an inorganic acid or an organic compound different than a carbohydrate and having an acidic character. Preferably, suitable inorganic and organic acids are those that are known as having no safety concern when applied in the pharma and food industry, for example those generally used for making a pharmaceutically acceptable acid-addition salt of an active pharmaceutical ingredient with basic character. Inorganic acid can be selected from e.g. sulfuric acid and its monotals (like monosodium sulphate), HCl, HBr, nitric acid, phosphoric acid and its mono- and disalts (like monosodium phosphate or disodium phosphate) or perchloric acid. Also preferably, suitable organic acids are alkanoic acids like formic acid, acetic acid or propionic acid, alkanoic diacids like oxalic acid, malonic acid or succinic acid, hydroxy acids like tartaric acid, lactic acid or malic acid, tricarboxylic acids like citric acid.

The basic non-carbohydrate component, optionally added in step b) or iiib), is typically an inorganic base such as a metal (for example alkali metal or alkali earth metal), hydroxide, carbonate or bicarbonate, advantageously a hydroxide, e.g. NaOH, KOH or Ca(OH)₂, preferably NaOH.

The pH of the aqueous solution obtained in step a) or i), or set in step b), iiia) or iiib), is not necessarily identical with the pH value that can be then measured in a 5 w/w % aqueous solution of the amorphous sialylated oligosaccharide after solidification according to step c) or iv) above, and it may differs in approximately max. +0.2-0.5 pH units, that is higher with approx. 0.2-0.5 pH units. The possible reasons may be, apart from the error order of the pH-electrode, that the composition of the samples slightly changes under the conditions of the solidification (e.g. evaporation of volatile acidic components, slight decomposition affecting the pH later, etc.) and/or the buffer capacity slightly changes. Empirically, when pH of the aqueous solution obtained in step a) or i), or set in step b) iiia or iiib), is measured between 3.1-5.6, the pH of the amorphous sialylated oligosaccharide obtained as a result of solidification in step c) or iv), when measured in its 5 w/w % aqueous solution, is around 3.4-6.0.

In step c), the aqueous solution obtained in step a) or b), whatever the case is, is solidified to an amorphous substance in a conventional way: e.g. spray-dried, freeze-dried or precipitated. Similarly, in step iv), the aqueous solution obtained in step iiia) or iiib), whatever the case is, is solidified to an amorphous substance in a conventional way: e.g. spray-dried, freeze-dried or precipitated.

A third aspect of the invention relates to an amorphous sialylated oligosaccharide obtained or obtainable by the method according to the second aspect.

Method for Improving the Chemical Stability and/or the Physical Properties of an Amorphous Sialylated Oligosaccharide

A fourth aspect of the invention is a method for preventing or reducing the isomerization in an amorphous sialylated oligosaccharide or HMO from aldose to ketose, the method comprising spray-drying or freeze-drying an aqueous solution of a sialylated oligosaccharide, the pH of which is not higher than 5.6, preferably lower than 5.0. The above method provides an amorphous sialylated oligosaccharide, the pH of which, when measured in its 5 w/w % aqueous solution, is not higher than around 6.0, preferably 5.3, that shows a reduced aldose-ketose isomerization.

The present inventors surprisingly found that the above rearrangement does not occur, or at least occurs in a significantly lower or reduced degree, in an amorphous state if the amorphous, e.g. spray-dried or freeze-dried, sialylated oligosaccharide or HMO is prepared from its aqueous solution where the pH is carefully adjusted. In this regard the chemical stability of the spray-dried or freeze-dried material can be substantially improved. This is achieved when an amorphous sialylated oligosaccharide or HMO is made from an aqueous solution of said sialylated oligosaccharide or HMO, the pH of the aqueous solution is not higher than 5.6, such as not higher than 4.1, 5.1 or 5.6, for example between 3.1-5.6, 3.1-5.1, 3.1-4.6, 3.1-4.1, 3.6-5.6, 3.6-5.1, 3.6-4.6, 3.6-4.1, 3.9-5.1, 4.1-5.1, 4.1-5.6, 3.9-4.6, 4.1-4.6, 4.6-5.6, 4.6-5.1 or 5.1-5.6. The amorphous sialylated oligosaccharide produced thereby has a pH, when measured in its 5 w/w % aqueous solution, of not more than around 6.0, such as not more than around 4.5, 5.1 or 5.6, for example between around 3.5-6, 3.5-5.6, 3.5-5.1, 3.5-4.5, 3.9-6, 3.9-5.6, 3.9-5.1, 3.9-4.5, 4.2-5.6, 4.5-5.6, 4.5-6.0, 4.2-5.1, 4.5-5.1, 5.1-6.0, 5.1-5.6 or 5.6-6.0.

Preferably, the amorphous sialylated oligosaccharide or HMO is made from an aqueous solution of said sialylated oligosaccharide or HMO, the pH of the aqueous solution is lower than 5.0, such as not higher than 4.1 or 4.8, for example between 3.1-4.8, 3.1-4.5, 3.1-4.1, 3.1-3.9, 3.1-3.6, 3.5-4.8, 3.5-4.5, 3.5-4.2, 3.5-3.9, 3.9-4.8, 3.9-4.5, 3.9-4.2, 4.2-5.0, 4.2-4.8 or 4.2-4.5. The amorphous sialylated oligosaccharide produced thereby has a pH, when measured in its 5 w/w % aqueous solution, of not higher than around 5.3, such as not higher than around 4.5 or 5.1, for example between around 3.5-5.3, 3.5-5.1, 3.5-4.8, 3.5-4.5, 3.5-4.2, 3.5-3.9, 3.9-5.3, 3.9-5.1, 3.9-4.8, 3.9-4.5, 3.9-4.2, 4.2-5.3, 4.2-5.1, 4.2-4.8, 4.2-4.5, 4.5-5.3, 4.5-5.1 or 4.5-4.8.

More preferably, the amorphous sialylated oligosaccharide or HMO is made from an aqueous solution of said sialylated oligosaccharide or HMO, the pH of the aqueous solution is not higher than 4.8, for example between 3.1-4.8, 3.1-4.5, 3.1-4.1, 3.1-3.9, 3.1-3.6, 3.5-4.8, 3.5-4.5, 3.5-4.2, 3.5-3.9, 3.9-4.8, 3.9-4.5, 3.9-4.2, 4.2-4.8 or 4.2-4.5. The amorphous sialylated oligosaccharide produced thereby has a pH, when measured in its 5 w/w % aqueous solution, of not higher than around 5.1, such as not more than around 4.5 or 4.8, for example between around 3.5-5.1, 3.5-4.8, 3.5-4.5, 3.5-4.2, 3.5-3.9, 3.9-5.1, 3.9-4.8, 3.9-4.5, 3.9-4.2, 4.2-5.1, 4.2-4.8, 4.2-4.5, 4.5-5.1 or 4.5-4.8.

Even more preferably, the amorphous sialylated oligosaccharide or HMO is made from an aqueous solution of said sialylated oligosaccharide or HMO, the pH of the aqueous solution is not higher than 4.5, for example between 3.1-4.5, 3.1-4.1, 3.1-3.9, 3.1-3.6, 3.5-4.5, 3.5-4.2, 3.5-3.9, 3.9-4.5, 3.9-4.2 or 4.2-4.5. The amorphous sialylated oligosaccharide produced thereby has a pH, when measured in its 5 w/w % aqueous solution, of not higher than around 4.8, such as not more than around 4.5, for example between around 3.5-4.8, 3.5-4.5, 3.5-4.2, 3.5-3.9, 3.9-4.8, 3.9-4.5, 3.9-4.2, 4.2-4.8, 4.2-4.5 or 4.5-4.8.

In one embodiment, the sialylated oligosaccharide is 3′-SL, its salt, preferably sodium salt, or mixture thereof. The amorphous 3′-SL, its salt, or mixture thereof is made from its aqueous solution, the pH of which is not higher than 5.6, such as not higher than 4.1, 5.1 or 5.6, for example between 3.2-5.6, 3.2-5.1, 3.2-4.6, 3.2-4.1, 3.6-5.6, 3.6-5.1, 3.6-4.6, 3.6-4.1, 3.9-5.1, 4.1-5.1, 4.1-5.6, 3.9-4.6, 4.1-4.6, 4.6-5.6, 4.6-5.1 or 5.1-5.6. The amorphous 3′-SL, its salt, or mixture thereof produced thereby has a pH, when measured in its 5 w/w % aqueous solution, of not more than around 6.0, such as not more than around 4.5, 5.1 or 5.6, for example between around 3.9-6, 3.9-5.6, 3.9-5.1, 3.9-4.5, 4.2-5.6, 4.5-5.6, 4.5-6.0, 4.2-5.1, 4.5-5.1, 5.1-6.0, 5.1-5.6 or 5.6-6.0. Preferably, the amorphous 3′-SL, its salt, or mixture thereof is made from its aqueous solution, the pH of which is lower than 5.0, such as not higher than 4.1 or 4.8, for example between 3.1-4.8, 3.1-4.5, 3.1-4.1, 3.1-3.9, 3.1-3.6, 3.5-4.8, 3.5-4.5, 3.5-4.2, 3.5-3.9, 3.9-4.8, 3.9-4.5, 3.9-4.2, 4.2-5.0, 4.2-4.8 or 4.2-4.5. The amorphous 3′-SL, its salt, or mixture thereof produced thereby has a pH, when measured in its 5 w/w % aqueous solution, of not higher than around 5.3, such as not higher than around 4.5 or 5.1, for example between around 3.5-5.3, 3.5-5.1, 3.5-4.8, 3.5-4.5, 3.5-4.2, 3.5-3.9, 3.9-5.3, 3.9-5.1, 3.9-4.8, 3.9-4.5, 3.9-4.2, 4.2-5.3, 4.2-5.1, 4.2-4.8, 4.2-4.5, 4.5-5.3, 4.5-5.1 or 4.5-4.8. More preferably, the amorphous 3′-SL, its salt, or mixture thereof is made from its aqueous solution, the pH of which is not higher than 4.8, for example between 3.1-4.8, 3.1-4.5, 3.1-4.1, 3.1-3.9, 3.1-3.6, 3.5-4.8, 3.5-4.5, 3.5-4.2, 3.5-3.9, 3.9-4.8, 3.9-4.5, 3.9-4.2, 4.2-4.8 or 4.2-4.5. The amorphous 3′-SL, its salt, or mixture thereof produced thereby has a pH, when measured in its 5 w/w % aqueous solution, of not more than around 5.1, such as not more than around 4.5 or 4.8, for example between around 3.5-5.1, 3.5-4.8, 3.5-4.5, 3.5-4.2, 3.5-3.9, 3.9-5.1, 3.9-4.8, 3.9-4.5, 3.9-4.2, 4.2-5.1, 4.2-4.8, 4.2-4.5, 4.5-5.1 or 4.5-4.8. Even more preferably, the amorphous 3′-SL, its salt, or mixture thereof is made from its aqueous solution, the pH of which is not higher than 4.5, for example between 3.1-4.5, 3.1-4.1, 3.1-3.9, 3.1-3.6, 3.5-4.5, 3.5-4.2, 3.5-3.9, 3.9-4.5, 3.9-4.2 or 4.2-4.5. The amorphous 3′-SL, its salt, or mixture thereof produced thereby has a pH, when measured in its 5 w/w % aqueous solution, of not higher than around 4.8, such as not more than around 4.5, for example between around 3.5-4.8, 3.5-4.5, 3.5-4.2, 3.5-3.9, 3.9-4.8, 3.9-4.5, 3.9-4.2, 4.2-4.8, 4.2-4.5 or 4.5-4.8. In other embodiment, the sialylated oligosaccharide is 6′-SL, its salt, preferably sodium salt, or mixture thereof. The amorphous 6′-SL, its salt, or mixture thereof is made from its aqueous solution, the pH of which is not higher than 5.2, such as not higher than 4.0 or 4.8, for example between 3.1-5.2, 3.1-4.8, 3.1-4.0, 3.6-5.2, 3.6-4.8, 3.6-4.0, 4.0-5.2, 4.0-4.8 or 4.8-5.2. The amorphous 6′-SL, its salt, or mixture thereof produced thereby has a pH, when measured in its 5 w/w % aqueous solution, of not more than around 5.6, such as not more than around 4.3 or 5.1, for example between around 3.4-4.3, 3.4-5.1, 3.4-5.6, 3.9-5.6, 3.9-5.1, 3.9-4.3, 4.3-5.6, 4.3-5.1 or 5.1-5.6. Preferably, the amorphous 6′-SL, its salt, or mixture thereof is made from its aqueous solution, the pH of which is lower than 5.0, such as not higher than 4.1 or 4.8, for example between 3.1-4.8, 3.1-4.5, 3.1-4.1, 3.1-3.9, 3.1-3.6, 3.5-4.8, 3.5-4.5, 3.5-4.2, 3.5-3.9, 3.9-4.8, 3.9-4.5, 3.9-4.2, 4.2-5.0, 4.2-4.8 or 4.2-4.5. The amorphous 6′-SL, its salt, or mixture thereof produced thereby has a pH, when measured in its 5 w/w % aqueous solution, of not higher than around 5.3, such as not higher than around 4.5 or 5.1, for example between around 3.5-5.3, 3.5-5.1, 3.5-4.8, 3.5-4.5, 3.5-4.2, 3.5-3.9, 3.9-5.3, 3.9-5.1, 3.9-4.8, 3.9-4.5, 3.9-4.2, 4.2-5.3, 4.2-5.1, 4.2-4.8, 4.2-4.5, 4.5-5.3, 4.5-5.1 or 4.5-4.8. More preferably, the amorphous 6′-SL, its salt, or mixture thereof is made from its aqueous solution, the pH of which is not higher than 4.8, for example between 3.1-4.8, 3.1-4.5, 3.1-4.1, 3.1-3.9, 3.1-3.6, 3.5-4.8, 3.5-4.5, 3.5-4.2, 3.5-3.9, 3.9-4.8, 3.9-4.5, 3.9-4.2, 4.2-4.8 or 4.2-4.5. The amorphous 6′-SL, its salt, or mixture thereof produced thereby has a pH, when measured in its 5 w/w % aqueous solution, of not more than around 5.1, such as not more than around 4.5 or 4.8, for example between around 3.5-5.1, 3.5-4.8, 3.5-4.5, 3.5-4.2, 3.5-3.9, 3.9-5.1, 3.9-4.8, 3.9-4.5, 3.9-4.2, 4.2-5.1, 4.2-4.8, 4.2-4.5, 4.5-5.1 or 4.5-4.8. Even more preferably, the amorphous 6′-SL, its salt, or mixture thereof is made from its aqueous solution, the pH of which is not higher than 4.5, for example between 3.1-4.5, 3.1-4.1, 3.1-3.9, 3.1-3.6, 3.5-4.5, 3.5-4.2, 3.5-3.9, 3.9-4.5, 3.9-4.2 or 4.2-4.5. The amorphous 6′-SL, its salt, or mixture thereof produced thereby has a pH, when measured in its 5 w/w % aqueous solution, of not higher than around 4.8, such as not more than around 4.5, for example between around 3.5-4.8, 3.5-4.5, 3.5-4.2, 3.5-3.9, 3.9-4.8, 3.9-4.5, 3.9-4.2, 4.2-4.8, 4.2-4.5 or 4.5-4.8.

A fifth aspect of the invention relates to a method for improving the chemical stability and/or the physical properties of an amorphous sialylated oligosaccharide or HMO, the method comprising solidifying, preferably spray-drying or freeze-drying, an aqueous solution of a sialylated oligosaccharide or HMO, the pH of which is not lower than 3.6, such as 3.9, and not higher than 5.6, lower than 5.0. The above method provides an amorphous sialylated oligosaccharide or HMO, the pH of which, when measured in its 5 w/w % aqueous solution, is not lower than 3.9 and not higher than around 6.0, preferably 5.3, and that shows a improved the chemical stability and/or physical properties.

The term “improving the chemical stability of an amorphous sialylated oligosaccharide” preferably means that an amorphous sialylated oligosaccharide is less prone to undergo chemical degradation or rearrangement reaction, when the pH of its aqueous solution, from which the amorphous sialylated oligosaccharide is produced, is within the above range, compared to an amorphous sialylated oligosaccharide that is made from its aqueous solution the pH of which is outside the above disclosed range. Therefore, the chemical stability that affects the shelf-life of the material is ameliorated. Example of chemical degradation is hydrolytic decomposition through breaking at least one of the interglycosidic linkages, typically of the sialidic linkage; example of chemical rearrangement is a rearrangement of an aldose to ketose.

The term “improving the physical properties of an amorphous sialylated oligosaccharide” preferably means that an amorphous sialylated oligosaccharide shows better physical properties when the pH of its aqueous solution, from which the amorphous sialylated oligosaccharide is produced, is within the above range, compared to an amorphous sialylated oligosaccharide that is made from its aqueous solution the pH of which is outside the above disclosed range. Examples of improved physical properties are lower propensity for phase change (e.g. vitrification, crystallization) or lower degree of agglutination.

The present inventors surprisingly discovered that the overall by-product formation (that is of the rearranged ulose and the hydrolysed product) in the solid (amorphous, e.g. spray-dried or freeze-dried) state of said sialylated oligosaccharide can be minimized. This is achieved when an amorphous sialylated oligosaccharide is made from an aqueous solution of said sialylated oligosaccharide, the pH of which is not lower than 3.6 and not higher than 5.6, such as 3.6-5.1, 3.6-4.6, 3.9-5.1, 4.1-5.1, 4.1-5.6, 3.9-4.6, 4.1-5.6, 4.6-5.6, 4.6-5.1 or 5.1-5.6. The amorphous sialylated oligosaccharide produced thereby has a pH, when measured in its 5 w/w % aqueous solution, of not lower than 3.9 and not higher than 6.0, such as 3.9-5.6, 3.9-5.1, 4.2-5.6, 4.5-5.6, 4.5-6.0, 4.2-5.1, 4.5-5.1, 5.1-6.0, 5.1-5.6 or 5.6-6.0.

Preferably, the amorphous sialylated oligosaccharide or HMO is made from an aqueous solution of said sialylated oligosaccharide or HMO, the pH of the aqueous solution is not lower than 3.6 and lower than 5.0, for example between 3.6-4.6, 3.6-4.2, 3.6-3.9, 3.9-4.6, 3.9-4.2 or 4.1-4.6. The amorphous sialylated oligosaccharide produced thereby has a pH, when measured in its 5 w/w % aqueous solution, of not lower than 3.9 and not higher than around 5.3, for example between around 3.9-5.3, 3.9-5.1, 3.9-4.8, 3.9-4.5, 3.9-4.2, 4.2-5.3, 4.2-5.1, 4.2-4.8, 4.2-4.5, 4.5-5.3, 4.5-5.1 or 4.5-4.8.

More preferably, the amorphous sialylated oligosaccharide or HMO is made from an aqueous solution of said sialylated oligosaccharide or HMO, the pH of the aqueous solution is not lower than 3.9 and lower than 5.0, for example between 3.9-4.6, 3.9-4.2 or 4.1-4.6. The amorphous sialylated oligosaccharide produced thereby has a pH, when measured in its 5 w/w % aqueous solution, of not lower than 4.2 and not higher than around 5.3, for example between around 4.2-5.1, 4.2-4.8, 4.2-4.5, 4.5-5.3, 4.5-5.1 or 4.5-4.8.

Even more preferably, the amorphous sialylated oligosaccharide or HMO is made from an aqueous solution of said sialylated oligosaccharide or HMO, the pH of the aqueous solution is not lower than 3.9 and not higher than 4.5, for example between 3.9-4.2. The amorphous sialylated oligosaccharide produced thereby has a pH, when measured in its 5 w/w % aqueous solution, of not lower than 4.2 and not higher than around 4.8, for example between around 4.2-4.5 or 4.5-4.8.

In one embodiment, the sialylated oligosaccharide is 3′-SL, its salt, preferably sodium salt, or mixture thereof. The amorphous 3′-SL, its salt, or mixture thereof is made from its aqueous solution, the pH of which is not lower than 3.9 and not higher than 5.6, such as 3.6-5.6, 3.6-5.1, 3.6-4.6, 3.6-4.1, 3.9-5.1, 4.1-5.1, 4.1-5.6, 3.9-4.6, 4.1-4.6, 4.6-5.6, 4.6-5.1 or 5.1-5.6. The amorphous 3′-SL, its salt, or mixture thereof produced thereby has a pH, when measured in its 5 w/w % aqueous solution, of around 4.2-6.0, such as around 4.2-5.6, 4.5-5.6, 4.5-6.0, 4.2-5.1, 4.5-5.1, 5.1-6.0, 5.1-5.6 or 5.6-6.0. Preferably, the amorphous 3′-SL, its salt, or mixture thereof is made from its aqueous solution, the pH of which is not lower than 3.9 and lower than 5.0, for example between 3.9-4.6, 3.9-4.1 or 4.1-4.6. The amorphous 3′-SL, its salt, or mixture thereof produced thereby has a pH, when measured in its 5 w/w % aqueous solution, of not lower than 4.2 and not higher than around 5.3, for example between around 4.2-4.9, 4.2-4.5, 4.5-5.3 or 4.5-4.9. More preferably, the amorphous 3′-SL, its salt, or mixture thereof is made from its aqueous solution, the pH of which is not lower than 3.9 and not higher than 4.6, for example between 3.9-4.1 or 4.1-4.6. The amorphous 3′-SL, its salt, or mixture thereof produced thereby has a pH, when measured in its 5 w/w % aqueous solution, of not lower than 4.2 and not higher than around 4.9, for example between around 4.2-4.5 or 4.5-4.9.

In other embodiment, the sialylated oligosaccharide is 6′-SL, its salt, preferably sodium salt, or mixture thereof. The amorphous 6′-SL, its salt, or mixture thereof is made from its aqueous solution, the pH of which is not lower than 3.6 and not higher than 5.2, such as between 3.6-5.2, 3.6-4.8, 3.6-4.0, 4.0-5.2, 4.0-4.8 or 4.8-5.2. The amorphous 6′-SL, its salt, or mixture thereof produced thereby has a pH, when measured in its 5 w/w % aqueous solution, of around 3.9-5.6, such as around 3.9-5.1, 3.9-4.3, 4.3-5.6, 4.3-5.1 or 5.1-5.6. Preferably, the amorphous 6′-SL, its salt, or mixture thereof is made from its aqueous solution, the pH of which is not lower than 3.6 and not higher than 4.8, such as not higher than 4.0 or 4.4, for example between 3.6-4.4, 3.6-4.0, 4.0-4.8, 4.0-4.4 or 4.4-4.8. The amorphous 6′-SL, its salt, or mixture thereof produced thereby has a pH, when measured in its 5 w/w % aqueous solution, of around 3.9-5.1, 3.9-4.8, 3.9-4.5, 3.9-4.3, 4.3-5.1, 4.3-4.8 or 4.3-4.5. More preferably, the amorphous 6′-SL, its salt, or mixture thereof is made from its aqueous solution, the pH of which is not lower than 3.6 and not higher than 4.4, such as not higher than 4.0, for example between 3.6-4.0 or 4.0-4.4. The amorphous 6′-SL, its salt, or mixture thereof produced thereby has a pH, when measured in its 5 w/w % aqueous solution, of around 3.9-4.8, 3.9-4.5, 3.9-4.3, 4.3-4.8 or 4.3-4.5.

Concerning all aspects disclosed above, in one embodiment, the sialylated oligosaccharide is a sialylated HMO, its acid addition salt or a mixture thereof. In a preferred embodiment, the sialylated HMO is 3′-SL, its acid addition salt or a mixture thereof, preferably sodium salt. In another preferred embodiment, the sialylated HMO is 6′-SL, its acid addition salt or mixture thereof, preferably sodium salt.

### EXAMPLES

### Example 1

3′-SL Na-salt was made by bacterial fermentation and isolated in accordance with e.g. WO 2017/152918. An aqueous stock solution was made the concentration of which was 5.3 g/100 g, pH: 4.8.

Series A: a part of the above aqueous stock solution was adjusted to pH of 3.6 by addition of 1 M HCl-solution. The so-obtained solution was freeze-dried to give a white amorphous powder. The pH of its 5 w/w % solution was 3.9.

Series B: a part of the above aqueous stock solution was adjusted to pH of 5.1 by addition of 1 M NaOH-solution. The so-obtained solution was freeze-dried to give a white amorphous powder. The pH of its 5 w/w % solution was 5.6.

Series C: a part of the above aqueous stock solution was adjusted to pH of 6.8 by addition of 1 M NaOH-solution. The so-obtained solution was freeze-dried to give a white amorphous powder. The pH of its 5 w/w % solution was 6.7.

Seven by one gram from each series, separately, was put into 4 ml glass vials and closed with screw lid containing teflon inliner (21 samples altogether). Three samples from each series were put into a 60° C. oven, and another three samples from each series were put into a 80° C. oven. One samples from each series was removed from the ovens after a time indicated below and kept in a freezer until analysis. The t=0 samples (one sample from each series) were kept in freezer during the entire time of the test.

The samples did not show phase change during investigation based on visual inspection.

The samples were analysed together after the longest incubation had finished by HPAEC-PAD on Carbopac PA-200 (3×250 mm) column using isocratic 75 mM NaOH+37.5 mM NaOAc eluent.

The table below shows the content of 3′-O-sialyl-lactulose, a ketose rearranged derivative of 3′-SL, in the samples (in area %, which is proportional with the amount).

| | sample A | sample B | sample C |
|---|---|---|---|
| t = 0 | 1.75 | 1.77 | 1.81 |
| 60° C., 2 weeks | 1.66 | 2.16 | 2.67 |
| 60° C., 4 weeks | 2.12 | 2.16 | 2.96 |
| 60° C., 6 weeks | 1.96 | 2.31 | 3.03 |
| 80° C., 1 weeks | 1.91 | 2.34 | 2.95 |
| 80° C., 2 weeks | 1.97 | 2.39 | 3.38 |
| 80° C., 4 weeks | 2.24 | 2.88 | 4.61 |

The data show that the higher the pH, the more the amount of the rearranged by-product under forced conditions. This suggests that the pH of the amorphous 3′-SL, when measured in its 5 w/w % solution, should not be higher than around 6 in order that a formation of significant amount of 3′-O-sialyl-lactulose is prevented/reduced in the solid sample when stored at ambient temperature.

In addition, the same study revealed that the lower the pH, the more the amount of the hydrolysed by-products (lactose and sialic acid) under forced conditions (data are not shown). In this regard, samples from series B contained the least overall amount of by-products (rearranged ketose+hydrolysis products). This suggests that the pH of the amorphous 3′-SL, when measured in its 5 w/w % solution, should not be higher than around 6 and not lower than around 4.2, in order that a formation of significant overall amount of rearranged and hydrolysis by-products is minimized in an amorphous sample when stored at ambient temperature.

### Example 2

6′-SL Na-salt was made by bacterial fermentation and isolated in accordance with e.g. US 2018/0002363. An aqueous stock solution was made the concentration of which was 9.1 g/100 g, pH: 5.4.

Series A: a part of the above aqueous stock solution was adjusted to pH of 3.1 by addition of 1 M HCl-solution. The so-obtained solution was freeze-dried to give a white amorphous powder. The pH of its 5 w/w % solution was 3.5.

Series B: a part of the above aqueous stock solution was adjusted to pH of 4.0 by addition of 1 M HCl-solution. The so-obtained solution was freeze-dried to give a white amorphous powder. The pH of its 5 w/w % solution was 4.3.

Series C: a part of the above aqueous stock solution was adjusted to pH of 4.8 by addition of 1 M HCl-solution. The so-obtained solution was freeze-dried to give a white amorphous powder. The pH of its 5 w/w % solution was 5.1.

Series D: a part of the above aqueous stock solution was adjusted to pH of 6.8 by addition of 1 M NaOH-solution. The so-obtained solution was freeze-dried to give a white amorphous powder. The pH of its 5 w/w % solution was 6.2.

Four by one gram from each series, separately, was put into 4 ml glass vials and closed with screw lid containing teflon inliner (16 samples altogether). Three samples from each series were put into a 80° C. oven. One sample from each series was removed from the oven after 1 week, 2 weeks and 4 weeks, respectively, and kept in a freezer until analysis. The t=0 samples were kept in freezer during the entire time of the stability test.

The samples did not show phase change during investigation based on visual inspection.

The samples were analysed together after the longest incubation had finished by HPAEC-PAD on Carbopac PA-200 (3×250 mm) column using isocratic 75 mM NaOH+37.5 mM NaOAc eluent.

FIG. 1 shows the content of 6′-sialyl-lactulose, a ketose rearranged derivative of 6′-SL, and the tendency of its formation in the samples (in area %, which is proportional with the amount).

The data show that the higher the pH, the more the amount of the rearranged by-product under forced conditions. This suggests that the pH of the amorphous 6′-SL, when measured in its 5 w/w % solution, should not be higher than around 5.5 in order that a formation of significant amount of 6′-O-sialyl-lactulose is prevented/reduced in the solid sample when stored at ambient temperature.

In addition, the same study revealed that the lower the pH, the more the amount of the hydrolysed by-products (lactose and sialic acid) under forced conditions (data are not shown). In this regard, samples from series B and C contained the least overall amount of by-products (rearranged ketose+hydrolysis products). This suggests that the pH of the amorphous 6′-SL, when measured in its 5 w/w % solution, should not be higher than around 5.5 and not lower than around 3.9, in order that a formation of significant overall amount of rearranged and hydrolysis by-products is minimized in an amorphous sample when stored at ambient temperature.

1. A method for preventing or reducing the isomerization in an amorphous sialylated oligosaccharide or its salt from aldose to ketose, the method comprising solidifying an aqueous solution of the sialylated oligosaccharide, wherein the pH of the aqueous solution is lower than 5.0.

2. The method according to claim 1, wherein the pH of the aqueous solution is not higher than 4.8.

3. The method according to claim 2, wherein the pH of the aqueous solution is not higher than 4.5.

4. The method according to claim 1, wherein the sialylated oligosaccharide is a sialylated human milk oligosaccharide (HMO).

5. A method for improving the chemical stability and/or the physical properties of an amorphous sialylated oligosaccharide or its salt, the method comprising solidifying an aqueous solution of the sialylated oligosaccharide, wherein the pH of the aqueous solution is not lower than 3.6 and lower than 5.0.

6. The method according to claim 5, wherein the improvement of the chemical stability is the prevention or reduction of the isomerization from aldose to ketose and hydrolytic decomposition.

7. The method according to claim 5 or 6, wherein the pH of the aqueous solution is not lower than 3.9 and lower than 5.0.

8. The method according to claim 7, wherein the pH of the aqueous solution ranges between 3.9 and 4.5.

9. The method according to claim 5, wherein the sialylated oligosaccharide is a sialylated human milk oligosaccharide (HMO).

10. The method according to claim 9, wherein the sialylated HMO is
3′-SL or its salt and the pH of its aqueous solution is not lower than 3.9, or
6′-SL or its salt.

11. An amorphous sialylated oligosaccharide or its salt, wherein the pH of said amorphous sialylated oligosaccharide, when measured in its 5 w/w % aqueous solution, is around 3.9 to around 5.3.

12. The amorphous sialylated oligosaccharide or its salt according to claim 11, wherein the sialylated oligosaccharide is a sialylated human milk oligosaccharide (HMO).

13. An amorphous sialylated oligosaccharide or its salt obtained or obtainable by the following method:
a) preparing an aqueous solution of said sialylated oligosaccharide,
b) if necessary, adding an acidic or a basic non-carbohydrate component to the aqueous solution obtained in step a) so that the pH is not lower than 3.6 and lower than 5.0, then
c) solidifying, preferably spray-drying or freeze-drying, the aqueous solution obtained in step a) or the pH adjusted solution obtained in step b),
thereby obtaining an amorphous sialylated oligosaccharide or its salt, wherein the pH of said sialylated oligosaccharide, when measured in its 5 w/w % aqueous solution, is not more than around 3.9 to around 5.3.

14. A method for making the amorphous sialylated oligosaccharide according to claim 11, comprising the steps of
a) preparing an aqueous solution of said sialylated oligosaccharide,
b) if necessary, adding an acidic or a basic non-carbohydrate component to the aqueous solution obtained in step a) so that the pH is not lower than 3.6 and lower than 5.0, then
c) solidifying, preferably spray-drying or freeze-drying, the aqueous solution obtained in step a) or the pH adjusted solution obtained in step b).

15. The method according to claim 14, wherein the sialylated oligosaccharide is a sialylated human milk oligosaccharide (HMO).

16. The method according to claim 15, wherein the sialylated HMO is 3′-SL or 6′-SL.

17. The method according to claim 4, wherein the sialylated HMO is 3′-SL or 6′-SL.

18. The method according to claim 12, wherein the sialylated HMO is 3′-SL or 6′-SL.

19. The method according to claim 1, wherein the solidifying comprises spray-drying or freeze-drying.

20. The method according to claim 5, wherein the solidifying comprises spray-drying or freeze-drying.
